(19) United States
(12) Patent Application Publication
(10) Pub. No.: US 2024/0398460 A1
(43) Pub. Date: Dec. 5, 2024

(54) CALIBRATION METHOD FOR CONTACT VERIFICATION SYSTEM

(71) Applicant: Adagio Medical, Inc., Laguna Hills, CA (US)

(72) Inventors: Brandon Ngai, Coto De Caza, CA (US); Kevin D. RUPP, Irvine, CA (US)

(21) Appl. No.: 18/667,042

(22) Filed: May 17, 2024

Related U.S. Application Data
(60) Provisional application No. 63/470,804, filed on Jun. 2, 2023.

(51) Int. Cl. A61B 18/02; A61B 18/00

(52) U.S. Cl. CPC A61B 18/02; A61B 2018/00077; A61B 2018/00095; A61B 2018/00136; A61B 2018/00351; A61B 2018/00577; A61B 2018/00648; A61B 2018/00714; A61B 2018/00797; A61B 2018/00821; A61B 2018/00839; A61B 2018/00875; A61B 2018/00904; A61B 2018/0212; A61B 2018/0262

(57) ABSTRACT

A system for verifying tissue contact between a catheter and tissue includes a processing device programmed and operable to compute a tissue contact value for each electrode pair on the catheter. Computing the tissue contact value is based on a measured impedance sample and an impedance baseline determined for each electrode pair. The system then classifies the tissue contact value based on a preestablished threshold. Methods of calibrating and verifying tissue contact are also described.

### CROSS-REFERENCE TO RELATED APPLICATIONS

This application claims priority to provisional application No. 63/470,804, filed Jun. 2, 2023, and entitled “CALIBRATION METHOD FOR CONTACT VERIFICATION SYSTEM.”

### BACKGROUND

### 1. Field of the Invention

Embodiments of the present invention relate to cryosurgery and more particularly to cryoablation systems adapted to confirm contact between a freeze section of the cryoablation catheter and the target tissue to be ablated.

### 2. Description of the Related Art

Cryoablation is a surgical technique for ablating tissue by cooling or freezing the tissue to a lethal degree. Cryoablation has the benefit of minimizing permanent collateral tissue damage and has applicability to a wide range of therapies including the treatment of heart disease.

Atrial flutter is a condition where the atria beat very quickly, but still evenly. Atrial fibrillation is not an uncommon form of heart disease. Atrial fibrillation occurs when anatomical obstacles in the heart disrupt the normally uniform propagation of electrical impulses in the atria. These anatomical obstacles (or conduction blocks) can cause the electrical impulse to degenerate into several circular wavelets that circulate about the obstacles. These wavelets, called “reentry circuits,” disrupt the normally uniform activation of the left and right atria.

Because of a loss of atrioventricular synchrony, people who suffer from atrial fibrillation and atrial flutter also suffer the consequences of impaired hemodynamics and loss of cardiac efficiency. These people are also at greater risk of stroke and other thromboembolic complications because of loss of effective contraction and atrial stasis.

One surgical method of treating atrial fibrillation by interrupting pathways for reentry circuits is the so-called “maze procedure,” which relies on a prescribed pattern of incisions to anatomically create a convoluted path, or maze, for electrical propagation within the left and right atria. The incisions direct the electrical impulse from the sinoatrial (SA) node along a specified route through all regions of both atria, causing uniform contraction required for normal atrial transport function. The incisions finally direct the impulse to the atrioventricular (AV) node to activate the ventricles, restoring normal atrioventricular synchrony. The incisions are also carefully placed to interrupt the conduction routes of the most common reentry circuits. The maze procedure has been found effective in treating atrial fibrillation. However, the maze procedure is technically difficult to perform. It also requires open heart surgery and use of a heart-lung machine, all of which is very expensive.

Maze-like procedures have also been developed utilizing energy sources to provide ablation energy to, for example, catheters, which can form lesions on the endocardium (the lesions being 1 to 15 cm in length and of varying shape) to effectively create a maze for electrical conduction in a predetermined path. The formation of these lesions by soft tissue coagulation (or ablation) can provide the same therapeutic benefits that the complex incision patterns that the surgical maze procedure presently provides, but without invasive, open heart surgery.

One lesion that has proven to be difficult to form with conventional devices is the circumferential lesion that is used to isolate the pulmonary vein to cure ectopic atrial fibrillation. Lesions that isolate the pulmonary vein may be formed within the pulmonary vein itself or in the tissue surrounding the pulmonary vein. Ablation of pulmonary veins is currently performed by placing a diagnostic catheter (such as Biosense Webster's Lasso™ circular ECG catheter, Irvine Biomedical's Afocus™ circular ECG catheter, or Boston Scientific Corporation's Constellation™ ECG catheter) into the pulmonary vein to be treated, and then ablating the pulmonary tissue adjacent to the distal end of the selected diagnostic catheter with a standard, commercially-available ablation catheter. The diagnostic catheter is used to determine if the lesion created by the ablation catheter has been successful in electrically isolating the pulmonary vein.

Some physicians may alternatively use a standard linear diagnostic catheter with 2-20 ECG electrodes to evaluate pre-ablation electrocardiogram (ECG) recordings. The physician then swaps the diagnostic catheter with a standard ablation catheter either through the same sheath, or in conjunction with the ablation catheter through a second sheath, ablating the area surrounding the pulmonary veins. The physician then swaps the ablation catheter with the diagnostic catheter to evaluate post-ablation ECG recordings.

The circumferential lesion must be iteratively formed by placing the ablation electrode into contact with a tissue region, ablating the tissue region, moving the ablation electrode into contact with another tissue region, and then ablating again. In a standard procedure, placement of the electrode and ablation of tissue may be repeated from 15-25 times to create the circumferential lesion necessary to electrically isolate the tissue. It is often difficult to form an effective, continuous circumferential lesion from a pattern of relatively small diameter lesions.

More recently, inflatable balloon-like devices that can be expanded within or adjacent to the pulmonary vein have been introduced.

US Patent Publication No. 2015/0018809 to Mihalik, for example, describes a method and system for cryotreatment and mapping of target tissue. The cryotreatment system may include a cryotreatment catheter, a mapping catheter including one or more mapping electrodes, and one or more temperature sensors located on the mapping catheter and/or the cryotreatment catheter. The cryotreatment catheter distal tip may be short enough to allow at least one mapping electrode to be positioned proximate the cryoballoon. Energy, such as radiofrequency energy, may be delivered to one or more mapping electrodes when one or more temperature sensors indicate a temperature of approximately 0° C. or below at one or more mapping electrode in order to thaw or prevent the formation of ice on the mapping electrodes when positioned proximate a cryoballoon during a cryotreatment procedure in order to recapture cardiac signals.

Although the above mentioned devices can be used to create various shaped lesions with some of better quality than others, the step of confirming whether the cryoenergy delivery section is in contact with the target tissue is still an important problem to be solved. Failure to make good tissue contact results in poor thermal conduction, and less predictable results.

Confirming whether the cryoenergy device is in contact with the target tissue is problematic because the diagnostic monitoring electrodes are either 1) on a separate device, or 2) offset a distance from the energy delivery section of the catheter. Consequently, the surgeon still does not know whether the energy delivery section is firmly contacting the target tissue. The surgeon may only estimate tissue contact based on fluoroscopy views, and the electrophysiological (EP) data transmitted from electrodes positioned nearby (but not within) the freeze zone. This type of instrument positioning is not straightforward, even for experienced surgeons.

Accordingly, a system and method that addresses the above mentioned challenges is still desired.

### SUMMARY

In embodiments of the invention, a tissue contact verification system provides real time feedback to a physician through a graphical user interface (GUI).

The system is operable to detect and process impedance signals measured at each catheter bipole. A contact state is determined based on changes in the impedance signal from a baseline. The system then classifies the contact state based on the degree or level of change from the baseline.

In other embodiments of the invention, the contact state is determined based on changes in the strength of specific frequency components that make up that signal. Without intending to being bound to theory, the inventors have found that when a certain bipole is found to be in good contact with tissue, the baseline impedance signal is modulated by the rhythm of the heart and respiratory rate.

In embodiments of the invention, a method for verifying tissue contact comprises advancing a distal section of the catheter through the vasculature of the patient such that the distal treatment section of the catheter is located adjacent to the tissue; separately computing a blood baseline value for a plurality of bipoles arranged along the distal treatment section; and classifying each bipole for tissue contact based on a real-time impedance signal of each bipole and the blood baseline value for each bipole from the computing step.

In embodiments of the invention, the method further comprises calculating an impedance offset for each bipole relative to a control or reference bipole, and computing a blood baseline value for each bipole based on the impedance offset.

In embodiments of the invention, the calculating step is based on measuring, ex vivo, impedance of the control bipole and each of the plurality of bipoles while suspended in a liquid (e.g., a saline solution). In embodiments of the invention, the impedance offset is a ratio.

In embodiments of the invention, a method for calibrating a tissue contact system comprises calculating an impedance offset for each bipole relative to a control bipole, and computing a blood baseline value for each bipole based on the impedance offset.

In embodiments of the invention, the method further comprises measuring, ex vivo, impedance of the control bipole and each of the plurality of bipoles while suspended in a liquid, and wherein the calculating the impedance offset is based on the ex vivo measuring step. In embodiments of the invention, impedance offset is a ratio.

In embodiments of the invention, a system for verifying tissue contact comprises a catheter comprising a flexible body and a distal treatment section; a plurality of bipoles arranged along the distal treatment section; and a processor. The processor is programmed to compute a blood baseline value for each bipole of the plurality of bipoles; and classify each bipole for tissue contact based on a real-time impedance signal of each bipole and the blood baseline value for each bipole.

In embodiments of the invention, the processer is further programmed to calculate an impedance offset for each bipole relative to a control bipole, and wherein the computing the blood baseline value is further based on the impedance offset.

In embodiments of the invention, the impedance offset is based on measuring, ex vivo, impedance of the control bipole and each of the plurality of bipoles while suspended in a liquid. In embodiments of the invention, the impedance offset is a ratio.

In embodiments of the invention, the processer is further programmed to compute the blood baseline for each bipole based on measuring impedance of the control bipole while suspended in blood in the patient.

In embodiments of the invention, the liquid in which the catheter is submerged for calibration is a saline solution.

In embodiments of the invention, each bipole is a pair of discrete electrodes.

In embodiments of the invention, the tissue is cardiac tissue selected from the group consisting of pulmonary vein openings, the atria or ventricles, and the cavo-tricuspid isthmus (CTI).

In embodiments of the invention, the processer is further programmed to display a tissue contact value for each bipole.

In embodiments, an animation shows whether the catheter is in contact with the tissue prior to ablation, and in preferred embodiments, whether each bipole is in contact with the tissue.

In embodiments of the invention, the catheter is further operable to ablate the tissue, and wherein the ablation is pulsed field and/or cryo-based. In embodiments the catheter is a multimodality ablation catheter operable to apply pulsed fields and cryo.

In embodiments of the invention, the control bipole is the distal most bipole.

In embodiments of the invention, the control bipole is the sole or only bipole required to be suspended in blood in the patient during calibration, and an impedance baseline for each bipole is computed based on the value of the control bipole in blood.

In embodiments of the invention, the catheter comprises at least 4 bipoles, and more preferably at least 16 bipoles.

### BRIEF DESCRIPTION OF THE DRAWINGS

The description, objects and advantages of the embodiments of the present invention will become apparent from the detailed description to follow, together with the accompanying drawings.

FIG. 1 is an illustration of a cryoablation system including a cryoablation catheter, according to an embodiment of the present invention;

FIG. 2 is a partial perspective view of a cryoablation catheter including a plurality of electrodes, according to an embodiment of the present invention;

FIG. 3 is an enlarged view of a portion of the catheter shown in FIG. 2;

FIG. 4 is a cross sectional view of the catheter taken along line 4-4 in FIG. 3;

FIG. 5A is an illustration of another cryoablation catheter according to an embodiment of the invention;

FIG. 5B is an enlarged side view of the distal section of the catheter shown in FIG. 5A;

FIG. 5C is an illustration of an endovascular catheterization to access the heart, according to an embodiment of the present invention;

FIG. 6 is an illustration of a distal section of a cryoablation catheter placed in a chamber of the heart, according to an embodiment of the present invention;

FIG. 7 is a flowchart showing a method for evaluating efficacy of a treatment procedure, according to an embodiment of the present invention;

FIG. 8 is a graph illustrating electrical activity between an electrode pair during various treatment stages of a cryoablation procedure, according to an embodiment of the present invention;

FIG. 9A is a partial view of a cryoablation catheter comprising a plurality of electrode sets, according to an embodiment of the present invention;

FIG. 9B is an enlarged view of an electrode set shown in FIG. 9A, according to an embodiment of the present invention;

FIG. 9C is an enlarged view of another electrode set shown in FIG. 9A, according to an embodiment of the present invention;

FIG. 10 is a schematic diagram of a system for verifying tissue contact, according to an embodiment of the present invention; and

FIG. 11 is a graph of electrical activity indicative of position of the distal section of the catheter relative to the tissue, according to an embodiment of the present invention;

FIG. 12 is a flowchart showing a method for classifying tissue contact, according to an embodiment of the present invention; and

FIGS. 13-17 depict a graphical user interface indicating status of each electrode in accordance with an embodiment of the invention.

### DETAILED DESCRIPTION

The following disclosure describes various embodiments of tissue contact verification systems with innovative features. These tissue contact verification systems are generally described in the context of cryoablation systems and cryoablation catheters. However, it should be understood that features of the disclosed systems can be applicable to other ablation technologies such as, for example, pulsed field ablation (PFA), microwave, ultrasound, HIFU, RF, etc. In addition, the embodiments of the present invention disclosed herein have applicability outside the ablation technologies and can be used with any medical devices to determine/confirm contact between the medical device and tissue. Moreover, while several components, techniques and aspects have been described with a certain degree of particularity, it is manifest that many changes can be made in the specific designs, constructions and methodology herein above described without departing from the spirit and scope of this disclosure.

It is to be understood that the embodiments of the invention described herein are not limited to particular variations set forth herein as various changes or modifications may be made to the embodiments of the invention described and equivalents may be substituted without departing from the spirit and scope of the embodiments of the invention. As will be apparent to those of skill in the art upon reading this disclosure, each of the individual embodiments described and illustrated herein has discrete components and features that may be readily separated from or combined with the features of any of the other several embodiments without departing from the scope or spirit of the embodiments of the present invention. In addition, many modifications may be made to adapt a particular situation, material, composition of matter, process, process act(s) or step(s) to the objective(s), spirit or scope of the embodiments of the present invention. All such modifications are intended to be within the scope of the claims made herein.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It will be understood that when an element is referred to as being “connected” or “coupled” to another element, it can be directly connected or coupled to the other element or intervening elements may be present. In contrast, if an element is referred to as being “directly connected” or “directly coupled” to another element, there are no intervening elements present.

It will also be understood that, although the terms first, second, etc. may be used herein to describe various elements, these elements should not be limited by these terms. These terms are only used to distinguish one element from another. Thus, a first element could be termed a second element without departing from the teachings of the present invention.

Language of degree used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. Additionally, numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

### System Overview

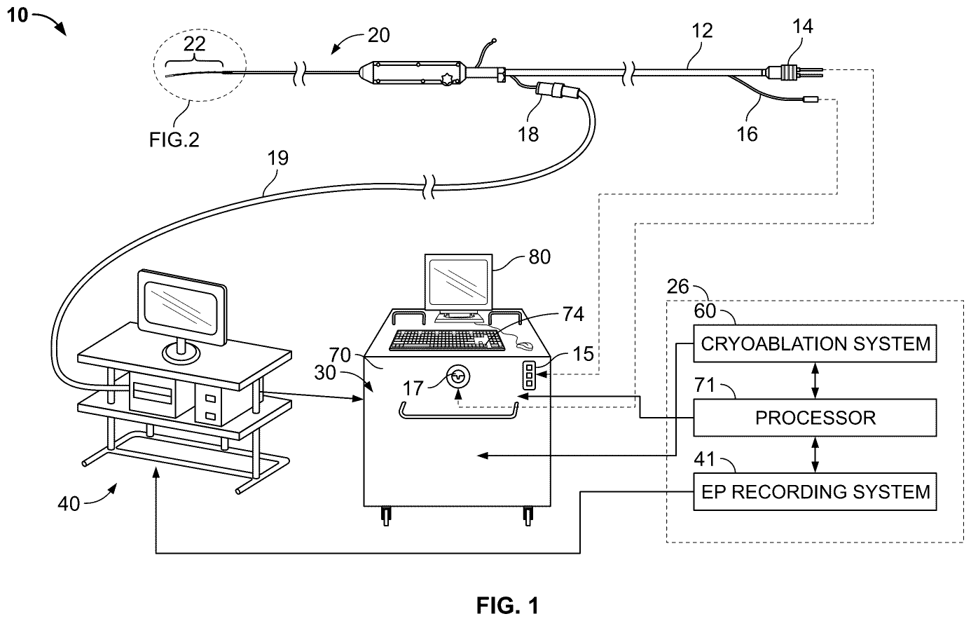

FIG. 1 illustrates a cryoablation system 10 according to an embodiment of the present invention, comprising a cryoablation console 30 and a cryoablation catheter 20 detachably connected to the console via a flexible cable 12. The flexible cable 12 terminates in a fluid connector 14 which is adapted to detachably attach with fluid-tight receptacle 17 on the console.

A temperature line 16 is also shown extending from cable 12. Temperature line 16 is detachably connectable to the console through the use of temperature ports 15.

In operation, as will be described in more detail herein, activation of the catheter causes a cryogen to be circulated through the distal treatment section 22 of the catheter. Tissue in contact with the distal treatment section 22 is cooled to a temperature sufficient to cause tissue necrosis. Temperature sensors or thermocouples present on the distal section (or elsewhere along the cryogen flow path) measure temperature. The temperature information is transmitted to the console 30 in real time, serving to measure and control cooling power during a surgical procedure. Examples and further details of cryoablation catheter systems are described in U.S. Pat. Nos. 7,410,484; 7,273,479; and 8,740,891; as well as International Patent Application No. PCT/US2014/056839, filed Sep. 22, 2014, entitled ENDOVASCULAR NEAR CRITICAL FLUID BASED CRYOABLATION CATHETER AND RELATED METHODS, the entire contents of each are incorporated herein by reference in their entirety for all purposes.

FIG. 1 also shows an electrophysiology (EP) recording system 40 adapted to record ECG signals. The EP recording system 40 is electrically coupled to cryoablation catheter 20 via connector 18 and flexible cable 19. Electrodes present on the distal treatment section 22 of cryoablation catheter 20, which shall be described in more detail herein, collect electrical information (e.g., cell voltage potentials for ECG readings, resistance, reactance, etc.) for determining catheter position, tissue contact, and/or treatment efficacy. The EP system may be a commercially available unit, such as, for example, the LabSystem PRO EP Recording System manufactured by Boston Scientific Inc. (Marlborough, MA). However, other EP systems may be used as described further herein.

The console 30 may house a variety of components (not shown) useful in operation of the cryoablation system including, without limitation, a controller, fluid tanks, valves, custom circuits, generators, regulators, pumps, etc. Examples of some of the components are described in U.S. Pat. Nos. 7,410,484; 7,273,479; and 8,740,891, the entire contents of each are incorporated herein by reference in their entirety for all purposes.

The console 30 includes a display 80 as shown in FIG. 1. In some embodiments, the display 80 is touch screen operable. The console 30 may also include other input devices such as, for example, a mouse and/or keyboard 74 to allow the user to input data and control the cryoablation system 10 and/or other devices.

The cryoablation system 10 preferably includes a processor 70, which is configured or programmed to control, for example, cryogen flowrate, pressure, and catheter temperatures as described herein. In some embodiments, as will be discussed further herein, the processor 70 can be programmed to receive electrical activity data (e.g. ECG recordings) from the EP recording system 40, and to determine tissue contact, catheter position, treatment efficacy, all of which may be indicated on display 80. It should also be understood that although the EP recording system 40 is shown physically separate from the cart 30, in other embodiments, the cart 30 includes or incorporates one or more of the components of the EP recording system (e.g. an amplifier, processor, circuits, etc.).

In embodiments of the present invention, the cryoablation system 10 includes a tissue contact verification system 26. In some embodiments, the tissue contact verification system 26 includes an EP recording module 41, a cryoablation system module 60, and a processor 71, all of which may be housed and interconnected within one convenient user-friendly console.

In other embodiments, the functional modules are physically separated but adapted to communicate with one another via wire or wireless transmission of data and information. And, as will be discussed further herein, the processor 71 can be programmed to automatically determine tissue contact and treatment efficacy based on data received from both the EP recording system or the EP recording module 41 and the cryoablation system module 60.

### Catheter Detail

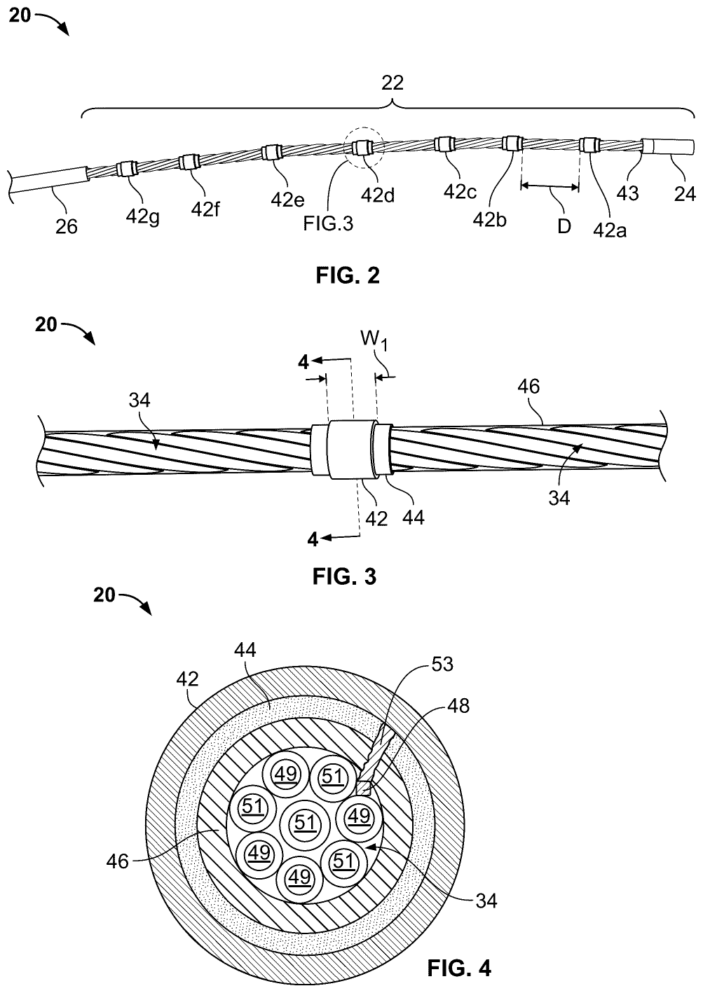

FIG. 2 shows an enlarged view of the distal treatment section 22 of the catheter 20 shown in FIG. 1. The distal section treatment section 22 contacts and freezes the target tissue. A plurality of electrodes 42 are disposed along the distal treatment section 22 for sensing and providing electrical information/signals at the treatment site during a treatment. In the embodiment depicted in FIG. 2, electrodes 42a-42g are included. As will be discussed in greater detail below, the tissue contact system measures/monitors electrical activity/signals between pairs of electrodes (e.g., between electrode 42a and electrode 42b, or between electrode 42a and electrode 42c, etc.) Using pairs of electrodes permits bipolar recording of the electrical activity/signals in the tissue at the treatment site. Bipoles may consist of adjacent pairs of electrodes (e.g., 42a-42b) or non-adjacent pairs of electrodes (e.g., 42a-42c). Additionally, in embodiments, each discrete bipole is monitored using a signal having a unique frequency to reduce interference between the different bipoles during monitoring.

As depicted in FIG. 2, the electrodes 42 are disposed in a lengthwise-spaced arrangement. Each electrode 42 is joined to a conducting wire that extends proximally to electrical connector 18. Notably, the electrodes 42 are shown situated within the energy delivery section (the distal treatment section 22), and are not included outside of the energy delivery section. As discussed further herein, placing the electrodes 42 within the treatment or energy delivery section provides more accurate electrical information in the treatment or energy delivery section 22 and therefore, a more accurate indication of the tissue contact than placing the electrodes outside of the treatment or energy delivery section 22, away from the tissue to be evaluated. In the embodiment shown in FIG. 2, the electrodes 42 are mounted on the freeze surface itself and measure/monitor the exact tissue location(s) within the freeze zone.

The spacing between the electrodes 42 may vary. In FIG. 2, for example, each electrode 42 is spaced a distance (D) from an adjacent electrode 42. The spacing distance (D) between the electrodes ranges from 0.5 to 25 mm. This spacing (D) can be uniform or it can vary between adjacent electrodes 42.

Additionally, the number of electrodes 42 may vary. In some embodiments, the number of electrodes 42 ranges from 2-20, more preferably ranges from 6-16, and in some embodiments, ranges from 8-12. The more electrodes 42 disposed along the distal treatment section 22, as will be discussed further herein, the more information that can be obtained from the treatment region for processing and analysis of catheter tissue contact and treatment efficacy.

The catheter 20 shown in FIG. 2 also includes a temperature sensor 43 (e.g., a thermocouple). Temperature sensor 43 is connected with the console 30 via the temperature line 16 (shown in FIG. 1) and temperature values may be utilized to adjust cooling power or energy during a treatment. Temperature sensors may also be disposed in line with the outflow and inflow of cryogen to measure a temperature difference between the inflow cryogen and outflow cryogen. The temperature difference may also be used to adjust cooling power to the tissue. With reference to FIG. 3, an enlarged view of a portion of the distal treatment section 22 depicted in FIG. 2 is shown. A thin, thermally conducting sleeve 46 (e.g., PET sleeve) is shown coaxially surrounding the bundle 34 of cryogen fluid transport tubes. An electrode 42 in the form of a ring is attached to the sleeve 46 via an epoxy layer 44. The electrodes 42 may be made of platinum or another electrically conducting material. Although the electrode 42 shown in FIGS. 2-4 has a ring-shape, the electrode may have other shapes. The width (W₁) of an electrode 42 may range from about 0.5 to 10 mm, and in embodiments, is preferably about 1-3 mm.

FIG. 4 shows a cross sectional view of the distal treatment section 22 of catheter 20 taken along line 4-4 in FIG. 3. A bundle 34 of cryogen fluid transport tubes form a circular array. The tube array 34 includes a plurality of inlet/delivery fluid transfer tubes 49 and a plurality of outlet/return fluid transfer tubes 51. In the embodiment shown in FIGS. 3-4, the plurality of inlet fluid transfer tubes 49 and a plurality of outlet fluid transfer tubes 51 are arranged in a twisted bundle. However, the tubes in the bundle 34 may be arranged in a variety of other configurations including, for example, positioning the inlet fluid transfer tubes 49 along the exterior of the bundle 34 and positioning the outlet fluid transfer tubes 51 on the interior of the bundle 34.

The inlet/delivery fluid transfer tubes 49 and outlet/return fluid transfer tubes 51 may be made of various materials. In some embodiments, the tubes are made of polyimide.

The size of the fluid transport tubes 49, 51 may vary. In some embodiments, the fluid transport tubes 49, 51 have an inner diameter in the range of approximately 0.02 to 0.1 inches. In some embodiments, the tubes have a wall thickness of approximately 0.002 inches.

During operation, the cryogen fluid is delivered to the catheter through a supply line from a suitable nitrogen source at a temperature of approximately −200° C. The cryogen is circulated through the multi-tubular 34 freezing zone provided by the exposed fluid transfer tubes 49, 51. The catheter may terminate at an endcap (e.g., endcap 24 as shown in FIG. 2). Endcap 24 may include an internal channel or chamber to fluidly connect the inlet fluid transfer tubes 49 to the outlet fluid transfer tubes 51.

In some embodiments, the cryogenic fluid utilized is nitrogen. In some embodiments, the nitrogen is supplied and maintained near it critical point (critical temperature of −147° C. and critical pressure of 492 psi). However, other cryogenic fluids may be utilized such as argon, neon, helium and others. In embodiments, the cryogen may be provided directly by the medical facility via a pressurized supply line. The system can include regulators and other devices to reduce or increase the pressure to the near critical pressure and temperature as desired. Other cryogenic fluid supply sources include, without limitation, a tank under pressure, mechanical pump, and a cryogen generator. Examples of such cryogenic fluid supply/generating sources are disclosed in U.S. Pat. No. 7,410,484, entitled “CRYOTHERAPY PROBE”, filed Jan. 14, 2004 by Peter J. Littrup et al.; U.S. Pat. No. 7,083,612, entitled “CRYOTHERAPY SYSTEM”, filed Jan. 14, 2004 by Peter J. Littrup et al.; U.S. Pat. No. 7,273,479, entitled “METHODS AND SYSTEMS FOR CRYOGENIC COOLING” filed Sep. 27, 2004 by Peter J. Littrup et al. U.S. Pat. Nos. 7,410,484, 7,083,612 and 7,273,479, the entire contents of each are incorporated herein by reference in their entirety for all purposes.

In embodiments, the nitrogen flow does not form gaseous bubbles inside the small diameter inlet fluid transfer tubes 49 and outlet fluid transfer tubes 51 under any heat load, so as to not create a phenomenon known as vapor lock, which limits/blocks the flow of nitrogen, which adversely affects the cooling power of the system. By operating the system at the near critical condition of nitrogen for at least an initial period of energy application, vapor lock is eliminated as the distinction between the liquid and gaseous phases disappears.

FIG. 4 also shows a conductive wire/element 48 in electrical connection with the electrode 42. Conductive wire/element 48 extends proximally to send electrical data/signals from the electrode 42 to the processor. The wire 48 may be soldered 53 or otherwise connected to the electrode 42 using other methods known to those of skill in the art (e.g., by use of electrically conducting adhesives, etc.).

In embodiments, and although not shown, the distal treatment section 22 of the catheter 20 may be deflectable. For example, either the fluid transport tubes themselves, or an ancillary element (e.g., and without limitation, a spine, stylet or shell member) can be used and can be, for example, made of a shape memory material such a nitinol, deflectable, or steerable to allow a user to manipulate the distal treatment section to make continuous and firm contact between the energy delivery elements and the target tissue. In some embodiments, the catheter configurations include substantial bends, or loops (e.g., full 360 degree loop-shape) which provide both the circumferential, as well as linear, ablations to mimic the surgical Maze procedure noted above. The catheters described herein may be manipulated (e.g., controllably deflected) to form ring-shaped lesions near or around the pulmonary vessel entries. Further examples of deflectable cryoablation catheters are described in PCT/US2015/024778, filed Apr. 7, 2015, entitled Endovascular Near Critical Fluid Based Cryoablation Catheter Having Plurality of Preformed Treatment Shapes, the entire contents of which are incorporated herein by reference in their entirety for all purposes.

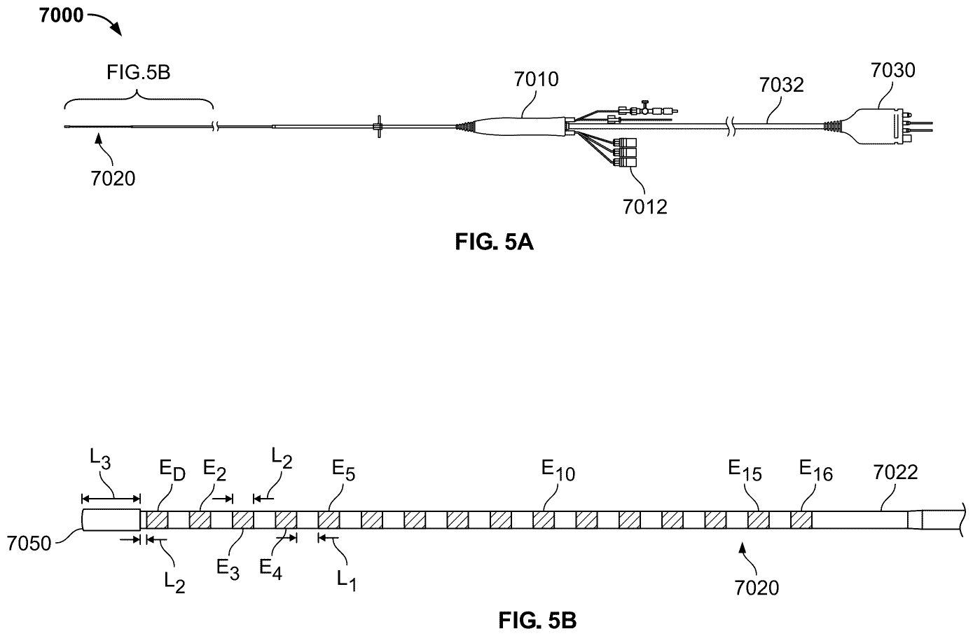

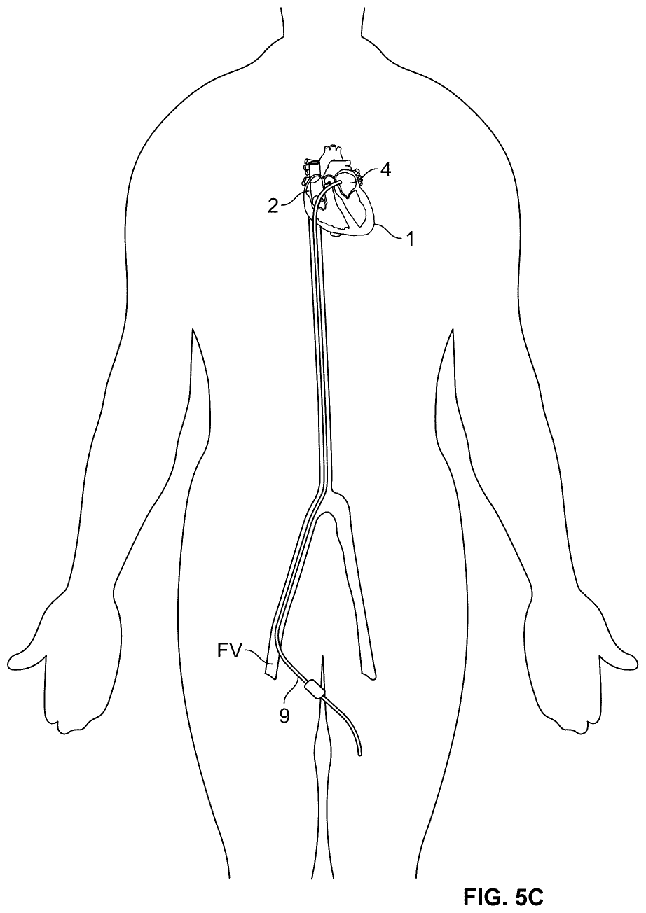

FIG. 5A shows another cryoablation catheter 7000 having a pulsed filed ablation modality in accordance with an embodiment of the invention. The catheter has a handle 7010, a distal section 7020 extending from the handle, a cryo-connector 7030, and an umbilical cord 7032 extending between the handle and cryo-connector 7030. Additionally, various connectors are coupled to handle. For example, FIG. 5A shows a thermocouple connector 7012 which is coupled to wires extending through the shaft of the catheter and for measuring/detecting temperature as described herein.

FIG. 5B is an enlarged view of the distal section 7020 of the catheter shown in FIG. 5A. The catheter has a shaft 7022 and plurality of axially spaced electrodes ED, E2, E3, E4, E5 . . . E10, E15, E16 commencing from tip 7050 which may also be an electrode in some embodiments. The electrodes serve a number of different functions including delivering pulsed fields for ablation (PFA) and for tissue contact verification, discussed herein.

The electrodes are shown spaced from one another by a distance (L1). The electrode spacing (L1) may vary. In embodiments, L1 ranges from 1-10 mm, more preferably 1-5 mm, even more preferably 2-4 mm. In one embodiment, L1 is about 3 mm.

Tip 7050 is shown spaced from the electrode E_{D} or E1 by a distance L2. In embodiments, L2 ranges from 1-10 mm, more preferably 1-5 mm, and in some embodiments 1-2 mm.

In embodiments, each electrode may have a width of L2, where L2 may range from 1-10 mm, more preferably 1-5 mm, and in some embodiments is about 2-4 mm. In one embodiment, L2 is about 3 mm.

In embodiments, the cap 7050 may have a width of L3, where L3 may range from 1-20 mm and more preferably 5-15 mm, and in some embodiments is about 8 mm.

In embodiments, the outer diameter of the electrodes ranges from 6 to 14 or, more preferably, 8 to 12 French. In embodiments, the thickness of each ring, band, or tubular shaped electrode is similar to that described above in connection with FIGS. 2-4.

In the embodiment shown in FIG. 5B, sixteen (16) ring electrodes are shown. Consequently, up to fifteen (15) electric fields may be generated spanning the spaces between adjacent electrodes as described herein. Or, in embodiments including tissue contact verification, 8 discrete (independent) bipoles. However, the number of electrodes may vary and more or less electrodes may be incorporated into the distal section of the catheter to generate the electric fields and discrete bipoles described herein, or to monitor electric signals of the tissue. An exemplary number of electrodes ranges between 2-40, more preferably 10-30, and in embodiments, 16-20.

In embodiments, opposite voltages are applied to adjacent electrodes to generate the electric fields. The voltages to the electrodes can be switched. Indeed, as described herein, a wide range of voltage patterns and pulse trains, pulse widths, and amplitudes may be applied to generate the electric fields suitable for PFA, and particularly, for PFCA. Examples of PFA and pulsed field cryoablation catheters are described in US Patent Publication No. 20220071681, filed Sep. 29, 2021 and PCT Publication No. WO2021/195311A1, filed Mar. 25, 2021, each of which is incorporated herein by reference in its entirety.

### Applications

The systems and methods described herein may be used in a variety of medical applications including, for example, cardiovascular applications. Examples of cardiovascular applications include, without limitation, treatment of arrhythmias such as atrial fibrillation and atrial flutter.

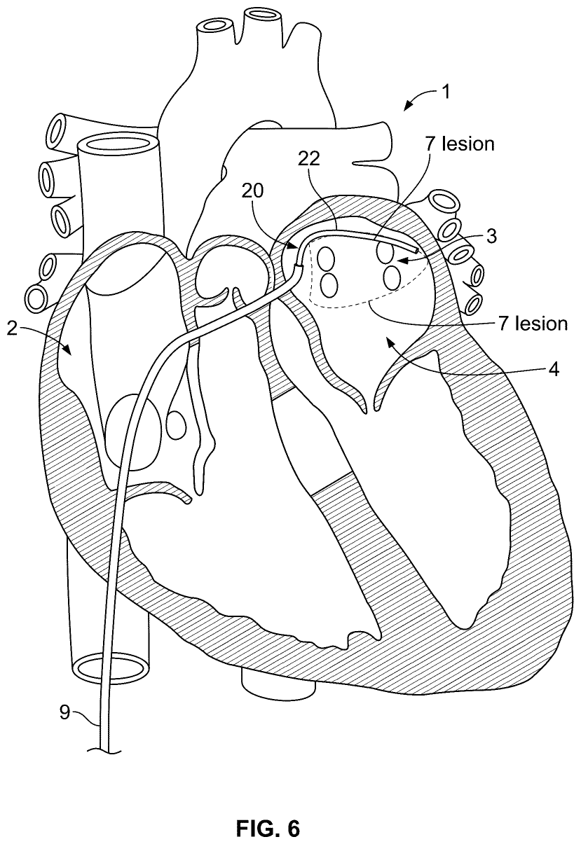

With reference to FIGS. 5C-6, a cardiovascular application for treating atrial fibrillation is shown. In particular, FIGS. 5C-6 illustrate creating an elongate, continuous lesion along the inner wall of the heart 1 in the left atrium 4. Creating such a lesion is known to be effective for treating various conditions such as, for example, atrial fibrillation because such continuous lesions electrically isolate the pulmonary veins 3. Further details of cryoablation systems and methodology are described in, for example, International Patent Application No. PCT/US2015/024778, filed Apr. 7, 2015, entitled Endovascular Near Critical Fluid Based Cryoablation Catheter Having Plurality of Preformed Treatment Shapes, the entire contents of which are incorporated herein by reference in their entirety for all purposes.

The example shown in FIGS. 5C-6 and following discussion tends to focus on embodiments for performing the left atrium lesion of the Cox maze VII procedure, however, the procedure for producing these lesions can be used to create other lesions in an around the heart and other organs. Additional lesions of the Cox maze VII procedure, as well as other variations of the Cox Maze treatments may be carried out using steps and devices described herein. Additional techniques and devices are described in international patent application nos. PCT/US2012/047484 to Cox et al. and PCT/US2012/047487 to Cox et al. corresponding to International Publication Nos. WO 2013/013098 and WO 2013/013099 respectively, the entire contents of each are incorporated herein by reference in their entirety for all purposes.

With reference to FIGS. 5C-6, one technique to reach the left atrium with the distal treatment section 22 of a catheter 20 is illustrated. A peripheral vein (such as the femoral vein FV) is punctured with a needle. The puncture wound is dilated with a dilator to a size sufficient to accommodate an introducer sheath, and an introducer sheath with at least one hemostatic valve is seated within the dilated puncture wound while maintaining relative hemostasis. With the introducer sheath in place, the guiding catheter or sheath 9 is introduced through the hemostatic valve of the introducer sheath and is advanced along the peripheral vein, into the target heart region (e.g., the vena cavae, and into the right atrium 2). Fluoroscopic imaging can be used to guide the catheter to the selected site.

Once in the right atrium 2, the distal tip of the guiding catheter 9 is positioned against the fossa ovalis in the intraatrial septal wall. A needle or trocar is then advanced distally through the guide catheter until it punctures the fossa ovalis. A separate dilator may also be advanced with the needle through the fossa ovalis to prepare an access port through the septum for seating the guiding catheter. The guiding catheter or sheath 9 thereafter replaces the needle across the septum and is seated in the left atrium through the fossa ovalis, thereby providing access for other devices through its own inner lumen and into the left atrium.

FIG. 6 shows an endocardial catheter 20 advanced through the guide catheter 9 and deployed as described herein to establish the desired lesion in the left atrium 4. The distal treatment section 22 of the endocardial catheter 20 is deflected within the endocardial space, preferably contacting the endocardial wall of the left atrium. This is illustrated in FIG. 6, where the distal treatment section 22 has been configured and deflected to cover the superior left atrial lesion 7, which partially encircles the left pulmonary veins 3. Although in the embodiment depicted in FIG. 6, the distal treatment section 22 is shown as only partially encircling the left pulmonary veins 3, in other embodiments, the distal treatment section 22 can be designed and configured to be manipulated to completely encircle the left pulmonary veins 3, thereby forming a single, continuous lesion that completely encircles the pulmonary veins.

### Tissue Contact and Ablation Verification

Described herein are systems, apparatuses and methods for verifying contact between the distal treatment section (e.g., distal treatment section shown in FIG. 2 or 5B) of a catheter and the target tissue to be treated (e.g., the tissue area corresponding to lesion 7 shown in FIG. 6).

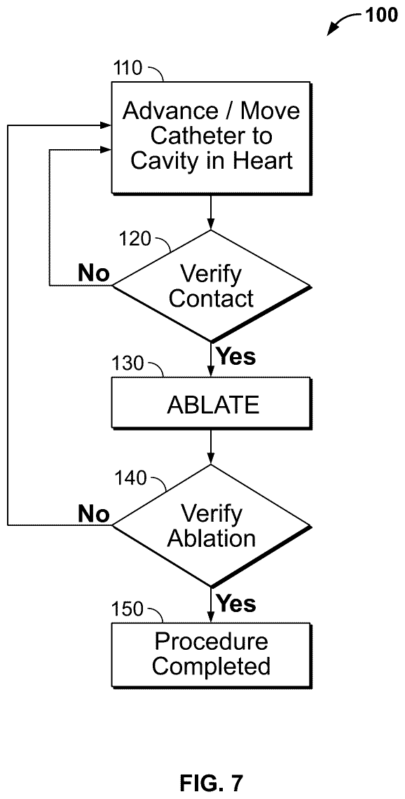

With reference to FIG. 7, a flowchart illustrates the steps of a method 100 according to an embodiment of the present invention, for verifying tissue contact between a catheter or other ablation device, for example, a cryoablation catheter as disclosed and described herein, and the target tissue. In this embodiment, the disclosed method is used for verifying/confirming contact between a cryoablation catheter and cardiac tissue.

Initially, and indicated by reference numeral 110, a cryoablation catheter as described herein is advanced into the applicable cavity of the organ (or chamber of the heart). For example, and without limitation, the distal treatment section 22 of catheter 20 may be advanced into the right atrium and positioned such that the most distal electrode is in contact with the ventricular end of the cavo tricuspid isthmus (CTI) for the treatment of atrial flutter, or into the left atrium and such that the distal section is in contact with the heart wall circumscribing the upper and lower pulmonary openings for the treatment of atrial fibrillation.

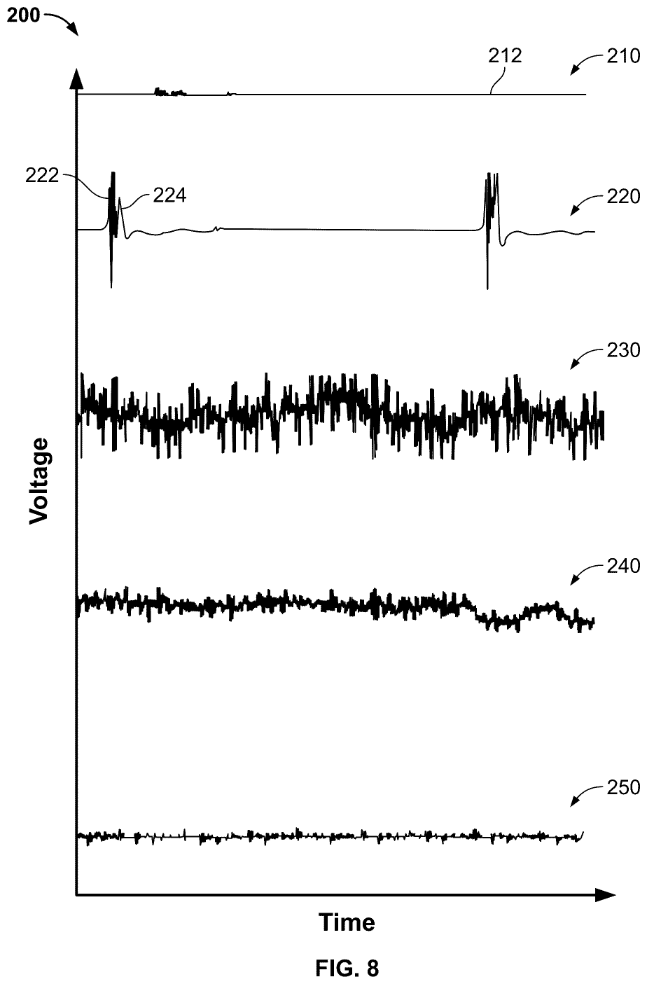

Step 120 recites to verify/confirm tissue contact by the distal treatment section 22. This step is carried out by sensing electrical activity through the electrodes 42 present on the distal treatment section 22 as discussed further herein in connection with FIG. 8, below. If the electrical information/signals received from the electrodes 42 indicate that suitable tissue contact has not been achieved, the catheter 20 is then moved to a new position in closer proximity to the target tissue or manipulated in its current position in an attempt to achieve better contact with the target tissue. Fluoroscopy may be used to assist the surgeon in determining positional information. Step 120 is repeated until tissue contact is confirmed by the electrical signals received from the electrodes 42.

Step 130 is freezing or otherwise ablating (e.g., RF, PFA, etc.) the target tissue to create the lesion. If the freezing is desired, the distal treatment section of the catheter is activated by circulating the cryogen through the distal treatment section. Tissue in contact with the distal treatment section is then ablated/frozen, causing necrosis.

Step 140 is to verify or confirm tissue necrosis following ablation. As discussed further herein in connection with FIG. 8, in embodiments, pretreatment data is compared with post treatment data.

If the ablation treatment is sufficient or within a threshold range, the treatment procedure may be deemed completed as indicated by step 150. If, on the other hand, the degree of ablation (or tissue necrosis) is not sufficient, the surgeon may return to step 110, and repeat the process as desired.

### Example 1

FIG. 8 is a graph 200 of ECG recordings (or signatures/signals) indicative of the electrical activity during a cryoablation procedure performed on healthy porcine tissue. The signatures were obtained by measuring electrical activity between a first electrode and a second electrode axially spaced from the first electrode (an electrode pair as discussed in more detail below).

Experiment Setup. A cryoablation system as described above in connection with FIGS. 1-4 was provided. The electrodes were connected to an ECG recorder system (GE Healthcare CardioLab II EP Recording System, Manufactured by GE Healthcare, USA.)

Initially, and with reference to signature 210, the distal section of the catheter was endovascularly advanced into the Right Atrium, near the Cavo Tricuspid Isthmus (CTI) of the heart, and spaced from the heart walls. The distal section of the catheter was surrounded by blood. The electrical activity recorded on the ECG system shows a relatively flat line 212, indicating that there was essentially no electrical activity measured arising from the beating heart. This flat curve 212 is anticipated because the blood does not conduct electrical current well from one electrode to the next electrode. This step may be used as a control signature/signal or baseline and compared to the ECG signatures/signals corresponding to the other stages of the treatment procedure.

Next, the distal treatment section (e.g. section 22 of FIGS. 1 and 2), is advanced or otherwise manipulated into contact with the target tissue (here, the atrium wall). A clear signature/signal 220 arises from the tissue contact as measured by the electrode pair and ECG system. Depolarization of the cells (or voltage vs. time) is measured by the electrode pair in direct contact with the tissue. The signature/signal is characterized by regular peaks 222, 224 indicative of the heartbeat. This implies the catheter distal treatment section portion between the first electrode to the second electrode (for example, electrodes 42a and 42b in FIG. 2), is in direct contact with the cardiac tissue. Tissue contact adjacent these two electrodes is thus verified/confirmed because a clear cardiac signature/signal 220 is measured by the electrode pair. In some embodiments, the distal treatment section 22 includes additional electrode pairs for analyzing tissue contact of additional portions of the catheter treatment section. Should no electrical activity be indicated after attempting to place the catheter against the target tissue, the catheter is further adjusted, deflected or moved. And an ECG signature may be analyzed for tissue contact. Thus, referring to FIG. 2, the graph of the ECG recordings depicted in FIG. 8, may be for electrodes 42a and 42b. Additional electrode pairs such as, for example, electrodes 42b and 42c (or more preferably independent pair 42c and 42d) may be connected to the ECG recording system to generate a graph similar to the graph in FIG. 8, of ECG signatures/signals for this pair of electrodes. In other embodiments, all of the electrode pairs (i.e., electrodes 42a and 42b, electrodes 42b and 42c, electrodes 42c and 42d, electrodes 42d and 42e, electrodes 42e and 42f, and electrodes 42f and 42g) may be connected to the ECG recording system to generate graphs of ECG signatures/signals for all of the electrode pairs on the distal treatment section. Connecting all of the electrode pairs to the ECG recording system permits a physician to obtain a complete picture of the entire distal treatment section contact with the target tissue.

### Ablation Activation

Now returning to FIG. 7, the method proceeds to ablation. In embodiments, and preferably without relocating or otherwise moving the catheter's distal treatment section, the cryoablation system and hence, the catheter is activated. In this embodiment, a near critical cryogen was circulated through the tube bundle in the distal treatment section of the catheter. The tissue in contact with the distal treatment section was ablated/frozen, forming a lesion Simultaneously, electrical activity between the first and second electrodes was recorded. This electrical activity is shown as signature 230 in FIG. 8. The signature 230 reflects the catheter as being activated because of the presence of the relatively high amplitude, and numerous scattered peaks, which are indicative of high noise.

Signature 240 reflects the electrical activity after halting the ablation. Although there is still some noise, the amplitude and number of peaks are minimized. Without being bound by theory, it is thought that the formation of ice in the tissue or surrounding the electrodes causes some electrical activity, and as the ice continues to melt or warm, the electrical activity and hence, the noise, is reduced.

The electrical information/signals received during these “Freeze On” and “Freeze Off” states is not only informative as to the conditions at the treatment site/target tissue, the information also increases safety. In particular, a physician can confidently know when to withdraw or move the cryoablation catheter from the target tissue subsequent to freezing the tissue. In stark contrast, failure to wait for the proper time to move the catheter away from the frozen tissue can cause collateral damage to the tissue because the catheter distal treatment section may still be stuck to the tissue during movement. With reference to signature 240 again, an electrical activity measured in the target tissue is shown to be minimal, or below a threshold value and thus, the surgeon may safely move the ablation catheter.

Next, and while the distal treatment section remains in the identical position as the preceding steps, the electrical activity is again measured/monitored. With reference to signature 250, little or no voltage is measured in comparison to peaks 222, 224 of signature 220. This is because tissue necrosis has occurred in the target area as a result of the cryoablation and necrosed tissue is a poor electrical conductor.

The above steps serve to confirm tissue ablation or tissue necrosis at the target site. A pre-treatment signature such as, for example, signature 220 is observed and compared to a post-treatment signature such as, for example, signature 250. Notably, the treatment section of the cryoablation catheter (and the monitoring electrodes thereon) remain in place until the physician confirms the entire target area has been ablated. This fixed-position technique is advantageous because the sensed electrical activity corresponds exactly to the entire length of the ablated tissue area, and the electrical activity is monitored immediately following ablation. Tissue necrosis and the absence of electrical activity along the entire length of target tissue can be confirmed, reflecting an effective ablation treatment.

### Arrhythmia Treatment Efficacy

Additionally, in some embodiments, a method further includes determining treatment efficacy for various arrhythmias including, for example, atrial fibrillation and atrial flutter. Whether the physician performs a Pulmonary Vein Isolation (PVI) approach with entrance and exit blocks for the treatment of atrial fibrillation or a bidirectional block approach for the treatment of atrial flutter, potentials are monitored across the ablation area to confirm successful blocking of abnormal electrical signals.

In embodiments, the cryoablation catheter (e.g., catheter 20 discussed above) is used as a diagnostic catheter (e.g., a circular mapping catheter) in addition to being used as an ablation catheter. For example, immediately following ablation in a PVI, the cryoablation catheter is advanced more distally inside a specific pulmonary vein to measure pulmonary vein potentials (voltage). Because the cryoablation catheter acts both as an ablation catheter and a diagnostic mapping catheter, there is no need for additional diagnostic mapping catheters (e.g., a lasso-type mapping catheter).

In other embodiments for determining treatment efficacy, intracardiac diagnostic catheters are provided in addition to the cryoablation catheter. For example, pacing catheters may be positioned at the coronary sinus artery and pace toward the left pulmonary veins, and/or the superior vena cava (SVC) and paced toward the right pulmonary veins. Additional diagnostic mapping catheters may be placed in the heart at, for example, the right atrium and the right ventricle. Examples of diagnostic catheters include but are not limited to: the deflectable Halo catheter (Cordis Webster, Baldwin Park, California), the decapolar catheter (Daig, Minnetonka, Minnesota), and the quadrapolar catheter (Cordis Webster, Baldwin Park, California). Details of intracardiac mapping following a PVI ablation are disclosed in, for example, Journal of Atrial Fibrillation, October-November 2013, Vol. 6, Issue-3, Electrophysiological Evaluation of Pulmonary Vein Isolation, by S. Kircher, P. Sommer.

Additionally, in embodiments, standard patch electrode ECG recordings (e.g., 12 lead waveforms and Holter monitoring) are evaluated before and after treatment to determine whether the disease or condition is alleviated. Follow up studies and monitoring may be performed at, for example, 2, 6, and 12 months following the surgery.

The tissue verification system described herein may have various embodiments. As shown in FIG. 1, for example, the tissue contact verification system may include a cryoablation catheter, a cryoablation console for driving and controlling the cooling power of the cryoablation catheter, and an ECG recording system which is physically separate from the cryoablation power console. A commercially available EP recorder may be provided which includes a standard receptacle for mating with the connector from the cryoablation catheter, and for receiving the electrical data during a procedure as described above. The EP recorder system may be operated to display the electrical ECG system arising from the cryoablation catheter electrodes. Depending on the GUI and features of the particular EP system, the data may be displayed, and further processed variously. The signatures may be recorded, displayed and compared. Examples of EP recording systems include without limitation the LabSystem PRO EP Recording System manufactured by Boston Scientific Inc. (Marlborough, MA). However, other EP systems may be used.

In another embodiment, the cryoablation system includes a computer programmed to receive electrical information (signals) from the EP recording system and to compute the tissue contact, freeze state, and treatment efficacy information. The cryoablation console may include an adapter or electrical receptacle to electrically communicate with the EP recording system. The computation step is automatic, and not subject to operating the EP recorder on a procedure by procedure basis to analyze data from the cryoablation catheter electrodes.

In another configuration, the EP recording system and the cryoablation system are provided as a standalone system (e.g., single console or cart). The system shares components such as, for example, a programmed processor for receiving the electrical activity from the electrodes on the distal treatment section. The processor may be programmed to process the electrical signals including without limitation, removing noise, identifying peaks and valleys, computing values of resistance, impedance, and capacitance, and comparing individual signatures with one another (such as baseline signatures to ablation signatures and baseline signatures to post-ablation signatures, etc.). Output may be displayed on a monitor in communication with the processor. Indicia may show levels of tissue contact, or length of the distal treatment section in contact with the target tissue based on which electrode pairs signals are indicating contact.

In embodiments, the cryoablation processor is programmed to not activate (“FREEZE-ON”) until tissue contact is verified. This step would avoid activating the catheter inconsistently or when there is incomplete contact with the tissue.

In some embodiments, the processor may be programmed to display a model catheter, the target tissue, color indicia representative of tissue contact, beacons, and symbols. In embodiments, the output is of a quantitative sort and based on tissue contact data, target tissue data, and design parameters of the cryoablation catheter in use (e.g., dimensions of the treatment section, bend angle, materials). For example, a percentage of the treatment section in contact with the target tissue may be computed and displayed. Automatic computation and programming has advantages because the output can be less susceptible to interpretation, estimation and error. Thus, the processor can be programmed to interpret the electrical signatures/signals received from the electrode pairs and based on the type of signal received (for example, signatures 210, 220, 230 and 240) indicate to the user the quality and extent of contact, whether the catheter is freezing, whether ice exists in the target tissue, etc. Based on the processor's interpretation, this information can be presented to the doctor graphically, pictorially, using text, etc. For example, the distal treatment section of the catheter, including the electrodes, can be recreated on the display and then based on the processor's interpretation of the received electrical signals, the portions of the distal treatment section that are confirmed in contact with the target tissue can be highlighted. For example, portions of the distal treatment section that are in good contact with the target tissue can be blue or green while portions of the distal treatment section that are not in contact with the target tissue can be white or red. Such a pictorial representation makes it easy for a doctor to quickly determine the contact conditions of the distal treatment section at the treatment location. Examples of a GUI depicting tissue contact are discussed further herein.

### Electrical Resistance

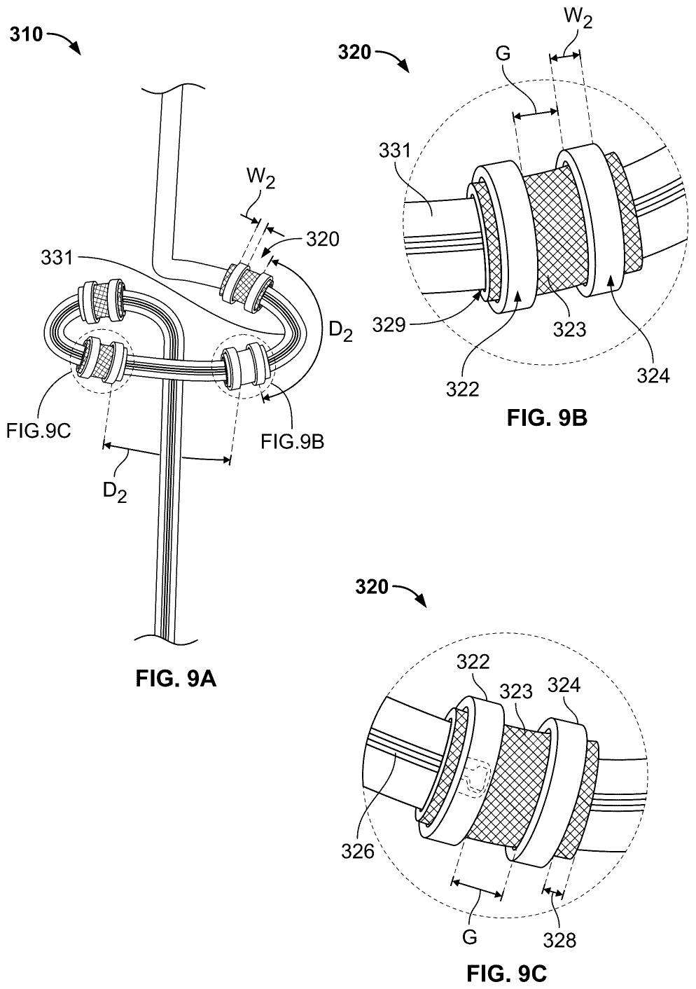

FIG. 9A illustrates a distal treatment section of a cryoablation catheter 310 according to another embodiment of the present invention. The catheter 310 comprises 4 electrode sets (e.g., set 320).

With reference to FIG. 9B, each electrode set 320 has 2 electrodes 322, 324. The electrodes are shown as ring-shaped, and are mounted on a polymeric sleeve 323, which is adhered with epoxy 329 to the catheter shaft. The width (W₂) of each electrode may vary and in embodiments ranges from approximately 0.5 to 2 mm, and in one embodiment is approximately 1.0 mm.

The gap between the individual electrodes can vary. In some embodiments the gap (G) is approximately 1.0 mm. Additionally, the electrode sets 320 are spaced apart from one another. In some embodiments, the spacing (D₂) between the sets 320 is approximately 0.5 to 2.0 inches and in particular embodiments, the spacing (D₂) between the electrode sets 320 is approximately 1.0 inch.

The catheter shaft 331 may be made as described above and include, for example, a bundle of fluid transport tubes that comprise inlet fluid transfer tubes and outlet fluid transfer tubes to provide cooling power to the target tissue. Shape memory and/or spine elements may be incorporated into the device to control deflection, angle and size of the deployment shape including a loop as shown in FIG. 9A.

With reference to FIG. 9C, one or more conducting wires 326 extend proximally from the electrodes to a connector in the handle (not shown), which can be interfaced to the EP recording system to transmit electrical activity between the electrodes and a computer processor.

In embodiments depicted in FIGS. 9A-9C, each electrode set 320 is a bipolar pair (i.e., each electrode set 320 includes a first electrode 322 and a second electrode 324). Using 2 electrodes in this bipolar configuration is advantageous as it permits elimination or reduction of the common noise between the two electrodes. This also reduces the Seebeck effect. Thus, using this bipolar electrode configuration results in an improved signal to noise ratio and hence, a cleaner electrical or ECG signal.

Although the embodiment of the invention depicted in FIG. 9A includes four (4) electrode sets 320, any number of electrode sets 320, may be used.

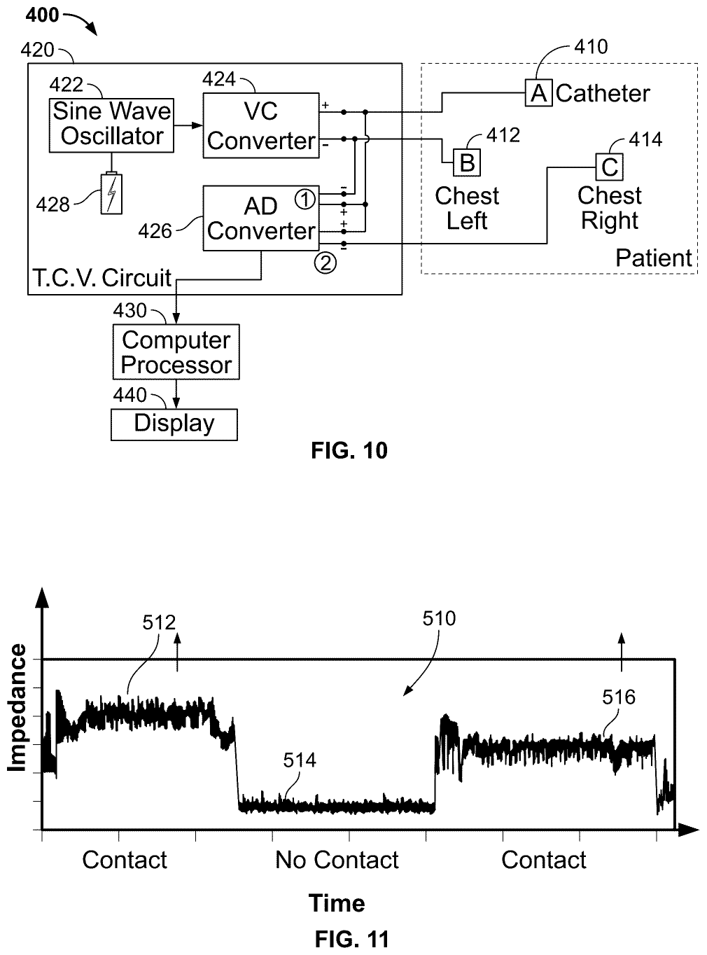

FIG. 10 is a schematic diagram of an embodiment a tissue contact verification system 400 incorporating a catheter 410 as shown in FIG. 9. The electrode sets 320 of the catheter 410 are connected to tissue contact verification (TCV) circuit 420. The TCV circuit 420 is shown having sine wave oscillator 422, and a voltage to current converter 424 for delivering current to the catheter electrode sets 320. A power source such as battery pack 428 may be included to deliver the initial DC current to wave oscillator 422. In embodiments, current from the current converter 424 is also sent to chest patch electrodes 412, 414 for recording baseline electrical activity and noise.

FIG. 10 also shows an analog to digital (AD) converter 426. The AD converter 426 receives electrical activity from the catheter, and left and right chest patch electrodes 412, 414 corresponding to Line (1) and Line (2) in FIG. 10.

As discussed further below, the digital information is sent to computer 430 for processing. Indicia of tissue contact between the catheter and tissue may be presented on display 440. An example of a circuit for measurement of electrical activity is described in U.S. Pat. No. 8,449,535 to Deno et al.

### Example 2

FIG. 11 is a graph showing a curve/signal 510 representative of electrical activity (e.g., resistance) arising from manually manipulating a cryoablation catheter similar to that described above with respect to FIG. 10 in use in porcine cardiac tissue in blood. The catheter was initially placed in contact with right atrial tissue, then it was moved so it was entirely floating in blood (no tissue contact), then again it was placed into contact with tissue. The positioning of the catheter was verified by fluoroscopy.

The curve/signal 510 includes peak 512, valley 514, and peak 516. Peaks 512, 514 of higher activity correspond to the applicable electrode region being placed in contact with the target tissue. Valley 514 corresponds to the catheter not making contact with the tissue. Peaks and valleys 510, 512 and 514 were measured from the same electrode pair that was moved into and out of contact with tissue. Without being bound by theory, this phenomena arises because tissue has a higher resistance than blood. When the current is sent through the tissue from one electrode to another, the resistance is higher than that of blood. This is reflected in peaks 512, 516.

In embodiments, reactance to the current sent through the tissue is monitored and impedance is computed. Impedance is defined in rectangular coordinates as Z=R+jX, where R is resistance, and jX is the reactance component. In embodiments, the impedance components (e.g., resistance and reactance) are separately measured and evaluated. In embodiments, a total impendence is monitored without separating out the individual components. While the above described circuit or system may be used to evaluate impedance the invention is not so limited. Other systems and circuits for generating and processing the current and signals may be incorporated into the present invention. Additional examples of circuits for measuring electrical activity are described in U.S. Pat. No. 8,449,535 to Deno et al.

In embodiments tissue contact may be confirmed, cryoablation performed, and treatment efficacy determined, with all steps carried out without repositioning the electrodes. Examples of details of a method and system for verifying tissue contact and treatment efficacy are described in U.S. Pat. No. 11,051,867, filed Jun. 13, 2018, incorporated herein by referee in its entirety.

### Alternate Embodiments

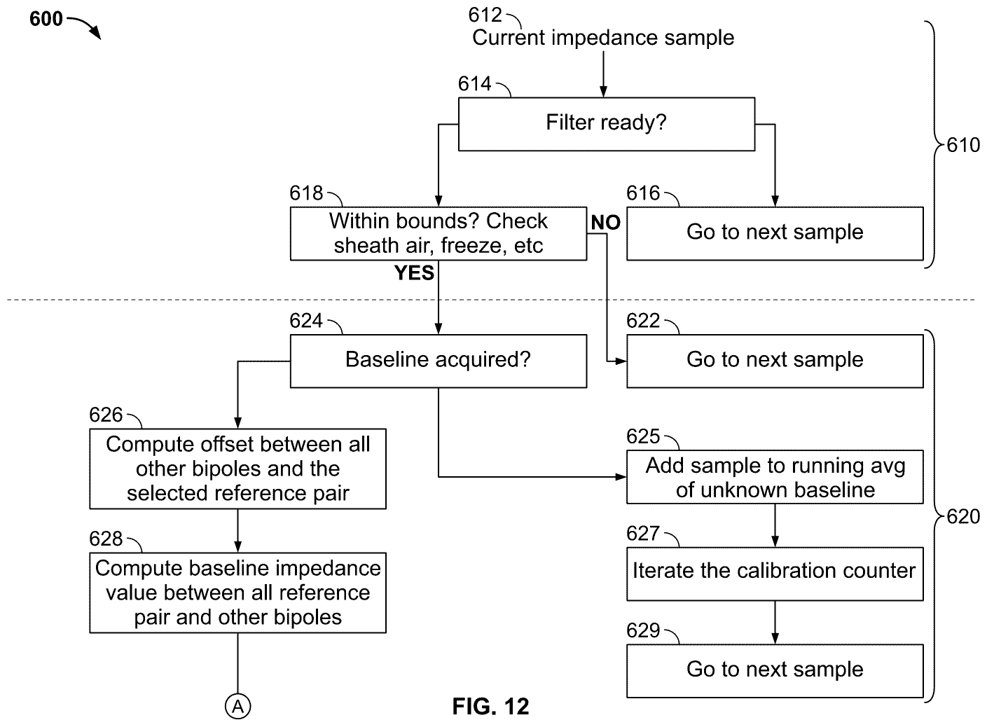

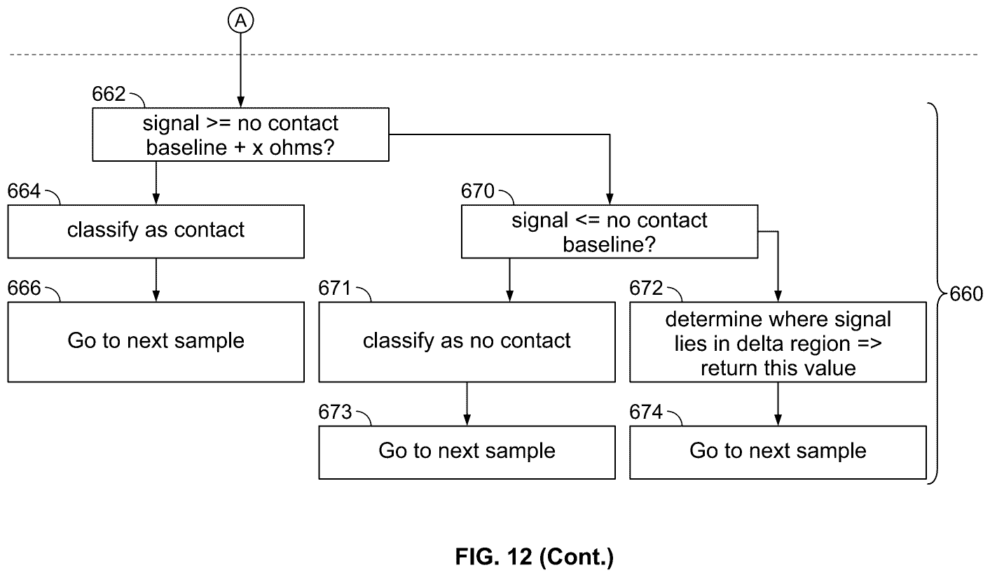

With reference to FIG. 12, another method 600 for verifying tissue contact in accordance with an embodiment of the present invention is shown. The method includes a filtering phase 610, a calibration phase 620, and a classification phase 660.

### Signal Preprocessing

With reference to the filtering phase 610, a low pass filter is initially applied to the impedance sample 612. The low pass filter serves to adequately reduce underlying noise on the impedance signal 612. Examples of filters include without limitation an exponential moving average (ema) filter and a Butterworth filter.

Filter parameters can be determined both theoretically and empirically to achieve the desired frequency response characteristics. The desired cut-off frequency of the filter can be determined through analysis of the baseline impedance signal in the frequency domain, and unwanted noise components can be identified and removed. As the filter is also highly dependent on the sampling rate of the impedance measurement hardware, filter constraints also rely on the desired latency of the entire system.

Additionally, in embodiments, a unique signal (e.g., unique frequency) is applied to each bipole to prevent signal interference between different bipoles.

Step 614 states filter ready. In step 614, the method queries whether there are enough samples to provide a filtered impedance output y(n). In embodiments, 20 to 40 samples are obtained per electrode pair, and preferably about 30 samples. In embodiments, as many samples are taken as possible up to a time limit. In embodiments, the time limit ranges from 5 to 10 seconds.

If there are a sufficient number of samples detected, then the method proceeds to the next step 618 to evaluate whether the filtered output falls within predetermined impedance limits, described below. However, if there are not enough samples taken, the method obtains the next impedance sample 616.

Step 618 applies a threshold-type filter to determine whether the processed output falls within a certain predetermined threshold impedance range, i.e., is within bounds. The bounds are based on empirical data of impedance values for air, freeze, or the sheath. If the filtered output is within bounds the method proceeds to the calibration phase 620. However, if the filtered output is greater than the threshold, the method proceeds to step 622 to obtain the next impedance sample. The threshold is typically dependent on the hardware and the catheter used in the procedure. An exemplary threshold value is 2500 ohms. However, in some embodiments of the invention, the threshold lies in the range of 2000-3000 ohms.

### Calibration

Step 624 of the calibration phase queries whether the baseline has been acquired. In embodiments of the invention, an ex vivo candidate baseline (e.g., saline-based impedance baseline) for each electrode pair is initially acquired by suspending all electrodes in a solution (e.g., 0.9% saline solution) without contacting the container. After a preestablished interval (which may vary to some degree based on the constraints arising from the particular impedance measurement hardware and filters), the impedance value of each electrode pair is recorded and the user is prompted to remove the catheter from the saline bath. A non-limiting exemplary range for the preestablished interval is 5-30 seconds, and preferably less than 10 seconds.

If, however, an ex vivo baseline for an electrode pair has not been acquired, the method proceeds to step 625 where the sample is recorded to a running average of unknown baseline data. The calibration counter is iterated (step 627). The method then proceeds to the next impedance sample for interrogation (step 629).

Next, and with reference to step 626, the system calculates an impedance offset or proportionality factor for each bipole for blood. To compute the impedance offset, the catheter is inserted into a target cavity in the patient. Examples of cavities include, without limitation, the heart atria or ventricles.

An electrode reference pair is then chosen. Selection of the reference pair may be automatically computed by the computer or by the user and based on anatomical location and orientation of the catheter bipoles. Optionally, imaging equipment typically available in the operating room can be used to confirm position, and whether an electrode pair is completely suspended in blood. If a certain pair is considered to be optimal for obtaining a floating impedance baseline, the system is programmed and operable to allow the user to select the pair as the reference pair.

For example, and with reference to FIG. 5B, the user may identify the most distal pair as the reference pair (ED-E2) and starting from the 2nd pair (E3-E4), until the 8th pair (E15-E16), the system is programmed and operable to calculate a percent impedance offset between each i^{th} pair and the most distal pair (ED-E2). In the catheter shown in FIG. 5B, eight (8) independent/discrete pairs of electrodes (E1-E2, E3-E4, E5-E6, E7-E8, E9-E10, E11-E12, E13-E14, E15-E16) are present. However, in other embodiments, more or less pairs of electrodes are arranged along the catheter and in some embodiments, electrodes are shared between the multiple bipoles.

In a preferred embodiment, the percent impedance offset for the i^{th} pair is computed as follows:

$${offset}_{ith pair} = \frac{saline {baseline}_{ith pair} - saline {baseline}_{reference pair}}{saline {baseline}_{reference pair}}$$

Next, and with reference to step 628, the blood or actual baseline impedance value is computed for each electrode pair based on the baseline impedance signal of the reference pair in blood and the offset value (e.g., offset ratio) for each electrode pair as computed above with reference to step 626. For example, in the embodiment shown in FIG. 5B, starting from the 2nd pair (E3-E4), until the 8th pair (E15-E16), the blood impedance baseline(s) are computed for the i^{th} pair using the saved offsets as described above and the actual impedance value of the reference pair in blood. In an embodiment, the blood baselines for each electrode pair (i) are computed as follows:

$$blood {baseline}_{i} = ( 1 + {offset}_{ith pair} ) \times blood {baseline}_{reference pair}$$

Once a baseline impedance value for each electrode pair has been computed, calibration is completed, and the classification phase 660, described below, may be commenced.

### Classification

After the calibration phase is completed, the method proceeds to the classification phase 660, which is performed per sample, and in real time.

With reference to step 662, the classification phase 660 compares the current impedance signal (e.g., y(n)ᵢₜₕₚₐᵢᵣ) to the corresponding baseline (e.g., bloodbaselineᵢₜₕₚₐᵢᵣ) established in step 628 during the calibration phase. In embodiments, a minimum contact impedance is established through in vivo studies and pre-ablation impedance analysis.

If the current impedance signal is greater than its baseline plus the preestablished threshold (e.g., a value in between 10-20 ohms), the contact state is classified as ‘contact’ with reference to step 664. The method then proceeds to the next sample as illustrated in step 666.

If the current impedance signal is less than its baseline plus the preestablished threshold), the contact state is classified as ‘no contact’ as illustrated in Step 671. The method then proceeds to the next sample as illustrated in step 673.

Optionally, with reference to step 670, in the case the current impedance signal is less than its baseline plus the preestablished threshold, the contact state is further evaluated in the step 672. A contact ratio is determined by computing the difference between the current impedance signal and the baseline, and then taking the ratio between the difference and the preestablished threshold as follows:

$$Contact ratio = \frac{signal - baseline}{threshold} ,$$

where the amount of electrode(s) in contact with the tissue corresponds to the ratio—the higher the ratio, the more tissue contact is estimated.

The method then proceeds to the next sample as illustrated in step 674.

While the calibration phase is typically performed once to calibrate the system, the classification phase may be repeated and updated in real time as the physician adjusts the location of the catheter in the cavity.

In another embodiment, calibration is performed entirely in vivo. The catheter is inserted into the heart chamber, and all electrodes are required to be suspended in blood without touching any surrounding myocardial tissue. This can be achieved by the physician through the means of other imaging systems, such as a fluoroscopy system, mapping system, and/or an intracardiac echocardiography system. The electrodes are suspended for a time interval sufficient to capture several samples and depends on the impedance measurement hardware and filter. An impedance baseline for each bipole in blood is therefore established.

In another embodiment of the invention, during classification of contact state, a windowed fast Fourier transform (FFT) is taken, and the power spectral density of the impedance signal is analyzed instead of processing the signal in the time domain as described above. When a bipole is in good contact with tissue, modulation by the heart and respiratory rhythm of the baseline impedance signal is observed. The algorithm detects changes in the power of these specific frequency bins and compares the strength of these components to the rest of the spectrum (i.e. the noise). By establishing a signal-to-noise (SNR) threshold between tissue contact and blood, a minimum contact SNR can be established, similar to the minimum contact impedance mentioned above.

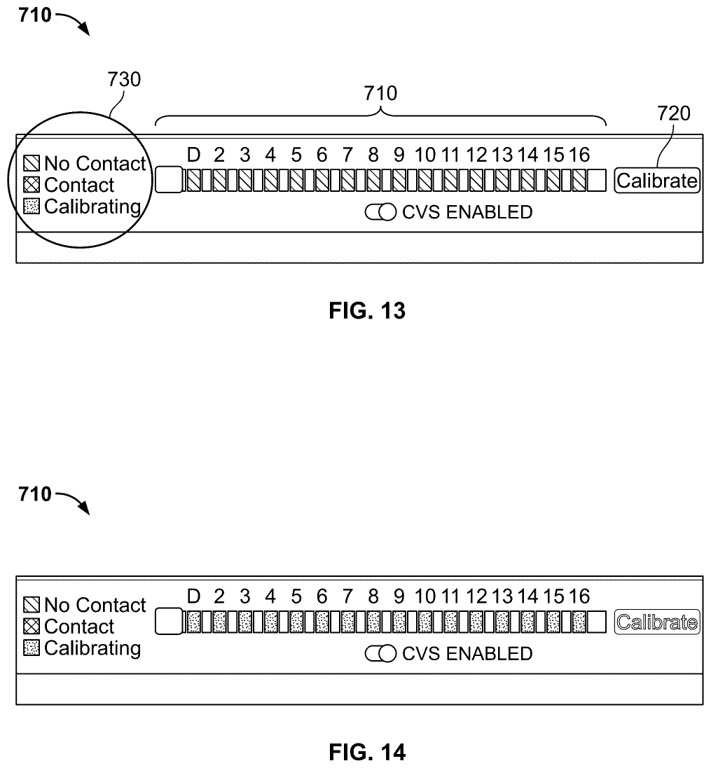

FIGS. 13-17 depict a graphical user interface (GUI) comprising a contact verification mode and window 700 in accordance with an embodiment of the invention.

With reference to FIG. 13, an illustration of a distal section of a catheter 710 comprises 16 electrodes (D-16), calibrate button 720, and legend 730 showing color or pattern codes to indicate status of the individual electrodes. Examples of color codes are white/clear (indicating no contact), blue/solid (indicating contact) and purple/diagonal lines (indicating calibrating). Indeed, the status indicators can vary widely from color, pattern, or density, etc. to indicate the different contact states of the electrodes.

With reference again to FIG. 13, all the electrodes are shown in a state of ‘no contact.’ Such a state represents the stage in the ex vivo calibration procedure when the catheter is initially submerged in the saline bath. Calibration button 720 is presented for activation.

With reference to FIG. 14, calibration button is shown greyed-out, indicating that it has been activated. All the electrodes are illustrated as in a state of ‘calibrating.’ Impedance samples for each bipole are taken and the impedance offset is computed as described above.

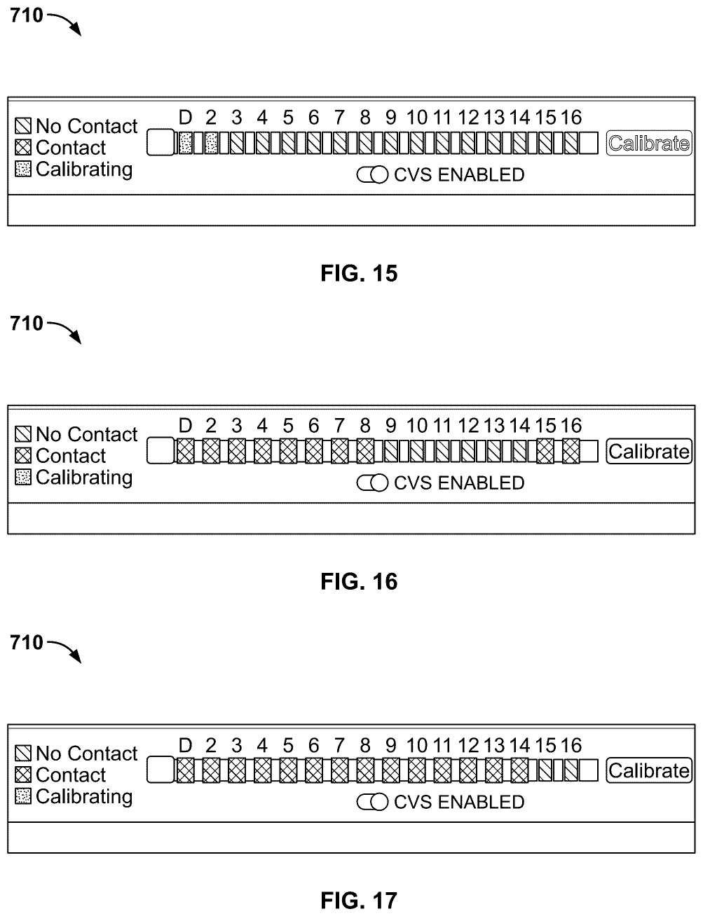

With reference to FIG. 15, only the distal bipole (D-2) are shown in a calibration state. Such a state represents the stage in the in vivo calibration procedure when only the distal bipole or control bipole is floated in blood. This impedance sample is then used to calculate the blood baseline for each bipole without requiring each bipole to be floated in blood.

With reference to FIG. 16, the catheter is shown in use to verify tissue contact. However, a gap exists along the length of the catheter where several bipoles (9-10, 11-12, and 13-14) indicate ‘no contact’. The physician can then adjust the positioning of the catheter in real time based on the displayed information.

With reference to FIG. 17, the physician has adjusted the catheter to place a continuous length of the catheter in contact with the tissue. A continuous length of contact is desirable for ablation, especially ablation to treat errant electrical signals in the heart. Gaps in the ablation can render the procedure ineffective. For at least this reason, the GUIs described herein provide useful tools for the physician to confirm tissue contact during a procedure.

Many modifications and variations of the present invention are possible in light of the above teachings. It is, therefore, to be understood that within the scope of the appended claims, the invention may be practiced otherwise than as specifically described.

1. A method for verifying tissue contact of a catheter with a tissue in a patient, the method comprising:
advancing a distal section of the catheter through the vasculature of the patient and into a candidate location for ablating the tissue;
obtaining a real-time impedance signal of each bipole of a plurality of bipoles arranged along the distal treatment section; and
classifying each bipole for tissue contact based on the real-time impedance signal of each bipole and a blood baseline value computed for each bipole.

2.-20. (canceled)

21. A cryoablation system for verifying tissue contact of a catheter with a tissue in a patient, comprising:
a catheter comprising a flexible body and a distal treatment section;
a plurality of bipoles arranged along the distal treatment section; and
a processor programmed to:
compute a blood baseline value for each bipole of the plurality of bipoles; and
classify each bipole for tissue contact based on a real-time impedance signal of each bipole and the blood baseline value for each bipole.

22. The system of claim 21, wherein the processer is further programmed to:
calculate an impedance offset for each bipole relative to a control bipole, and wherein computing the blood baseline value is further based on the impedance offset.

23. The system of claim 22, wherein the impedance offset is based on measuring, ex vivo, impedance of the control bipole and each of the plurality of bipoles while suspended in a liquid.

24. The system of claim 23, wherein the impedance offset is a ratio.

25. The system of claim 24, wherein the impedance
$${offset}_{ith pair} = \frac{saline {baseline}_{ith pair} - saline {baseline}_{reference pair}}{saline {baseline}_{reference pair}} .$$

26. The system of claim 22, wherein the processer is further programmed to compute the blood baseline for each bipole based on measuring impedance of the control bipole while suspended in blood in the patient.

27. The system of claim 26, wherein the
$$blood {baseline}_{i} = ( 1 + {offset}_{ith pair} ) * blood {baseline}_{reference pair} .$$

28. The system of claim 23, wherein the liquid is a saline solution.

29. The system of claim 21, wherein each bipole is a pair of discrete electrodes.

30. The system of claim 21, wherein the tissue is cardiac tissue is selected from the group consisting of pulmonary vein openings, the atria or ventricles, and the cavo-tricuspid isthmus (CTI).

31. The system of claim 21, wherein the processer is further programmed to display a tissue contact value for each bipole.

32. The system of claim 21, wherein the catheter is further operable to ablate the tissue.

33. The system of claim 32, wherein the ablation is cryo and/or electroporation-based.

34. (canceled)

35. (canceled)

36. (canceled)

37. (canceled)

38. (canceled)

39. (canceled)

40. The system of claim 21, wherein the control bipole is the sole/only bipole required to be suspended in blood in the patient for calibrating the system.

41. The system of claim 21, further comprising selecting the control bipole.

42. The system of claim 41, wherein the control bipole is automatically selected by the computer based on live image data.

43. (canceled)

44. The system of claim 21, wherein the tissue contact verification system is operable to determine a contact state for a plurality of electrodes and a non-cardiac tissue, and optionally, the tissue is a tissue wall of the respiratory and gastrointestinal systems.
